(19) United States
(12) Patent Application Publication
(10) Pub. No.: US 2009/0099144 A1
(43) Pub. Date: Apr. 16, 2009

(54) ORAL CONTRACEPTION (LONG-CYCLE REGIMEN AND 21-DAYS-REGIMEN WITH 30 MG ETHINYLESTRADIOL AND 2 MG DIENOGEST) WITHOUT TREATMENT OF THROMBOSIS/HEMOSTASIS

(76) Inventor: Katrin Mittmann, Jena (DE)

Correspondence Address: STRIKER, STRIKER & STENBY, 103 EAST NECK ROAD, HUNTINGTON, NY, 11743, US

(21) Appl. No.: 12/248,583

(22) Filed: Oct. 9, 2008

Related U.S. Application Data
(60) Provisional application No. 60/979,210, filed on Oct. 11, 2007.

(51) Int. Cl. A61K 31/565

(52) U.S. Cl. 514/170

(57) ABSTRACT

The kit for long-cycle administration of a monophasic oral contraceptive preparation without affecting blood pressure advantageously consists of 84 daily dose units, which each contains a combination of 2.0 mg of dienogest and 0.030 mg of ethinyl estradiol, and 7 daily dose units that do not contain any active contraceptive agent or contain a placebo. During long-cycle administration over the course of a year using four 91-day cycle kits, no significant differences in the resulting hemostatic variables in comparison to those obtained during conventional administration with 28-day cycle kits, which each contain 21 daily dose units each containing the combination and 7 daily dose units without the combination or with a placebo, were observed at any time.

### CROSS REFERENCE

The invention described herein below is also described in U.S. Provisional Patent Application Ser. No. 60/979,210, filed on Oct. 11, 2007. The aforesaid US Provisional Patent Application provides the basis for a claim of priority of invention for the invention claimed and described herein below under 35 U.S.C. 119 (e).

### BACKGROUND OF THE INVENTION

The conventional regimen of oral contraceptives (OC) which typically consists of 21 days of administration of an estrogen/progestogen combination and a subsequent hormone-free interval of 7 days was introduced about 45 years ago and is still the standard of hormonal control of fertility. However, there is no medical reason for the regular withdrawal bleeding associated with the rapid decline of serum levels of contraceptive steroids during the pill-free week. This regimen was chosen to simulate normal cyclic events and to facilitate the general acceptance of this new contraceptive method (Wiegratz I, Kuhl H, Long-cycle Treatment With Oral Contraceptives, Drugs 2004, 64: pp. 2447-62).

On the other hand, the regular cyclic fluctuations in the serum concentrations of the contraceptive steroids, i.e., an increase during the first days of administration up to a steady state followed by a rapid fall to baseline levels in the hormone-free week (Jung-Hoffmann C, Kuhl H, Pharmacokinetics and Pharmacodynamics of Oral Contraceptive Steroids: Factors Influencing Steroid Metabolism, Am. J. Obstet. Gynecol. 1990, 163: pp. 2183-97), are associated not only with changes in many metabolic parameters, particularly hepatic proteins, but also with possible somatic and psychic complaints (Wiegratz I, Kuhl H, ibid; Sulak P J, Kuehl T J, Ortiz M, Shull B L, Acceptance of altering the standard 21-day/7-day oral contraceptive regimen to delay menses and reduce hormone withdrawal symptoms, Am. J. Obstet. Gynecol. 2002, 186: pp. 1142-9).

Therefore, omission of the hormone-free interval and continuous use of OCs for several weeks or months has been individually practiced for many years by women suffering from cycle-dependent symptoms or menses-related disorders. Various international surveys have indeed revealed that the majority of women prefer the extended regimen to the conventional use (Wiegratz I, Kuhl H, ibid; Wiegratz I, Hommel H H, Zimmermann T, Kuhl H, Attitudes of German Women and Gynecologists Towards Long-cycle Treatment With Oral Contraceptives, Contraception 2004, 69: pp. 37-42).

To replace this off-label use of OCs by treatment with approved OC regimens, long-cycle preparations have been developed to reduce the frequent regular bleeding during conventional use of OCs. Clinical studies have been carried out to investigate the effect of continuous use of OCs for 7, 9, 12 or 24 weeks without any hormone-free interval. After these time-periods, most investigators have inserted a 7-day hormone-free break as a compromise in order to cause withdrawal bleeding (Wiegratz I, Kuhl H, ibid).

Although many advantages of the extended OC cycles are obvious or probable, no data are available concerning the risks associated with long-cycle regimens. Thromboembolic disease is one of the most important complications of OC use. The relative risk of deep vein thrombosis (DVT) rises particularly during the first months of use, suggesting a major role of predisposition and risk factors (Helmerhorst F M, Rosendaal F R, Vandenbroucke J P, Venous Thromboembolism and the Pill, The WHO Technical Report on Cardiovascular Disease and Steroid Hormone Contraception: State-of-the-art, Hum. Reprod. 1998, 13: pp. 2981-3; Herings R M C, Urquhart J, Leufkens H G M, Venous Thromboembolism Among New Users of Different Oral Contraceptives, Lancet 1999, 354: pp. 127-128; Bloemenkamp K W M, Rosendaal F R, Helmerhorst F M, Vandenbroucke J P, Higher Risk of Venous Thrombosis During Early Use of Oral Contraceptives in Women With Inherited Clotting Defects, Arch. Intern. Med. 2000, 160: pp. 49-52; and Lidegaard O, Edström B, Kreiner S, Oral Contraceptives And Venous Thromboembolism: A Five-year National Case-control Study, Contraception 2002, 65: pp. 187-96). The risk was found to be dependent on the dose of ethinyl estradiol (EE) (Gomes M P V, Deitcher S R, Risk of Venous Thromboembolic Disease Associated with Hormonal Contraceptives and Hormone Replacement Therapy, Arch. Intern. Med. 2004, 164: pp. 1965-1976; Tchaikovski S, Tan G, Rosing J, Venous Thrombosis and Oral Contraceptives: Current Status, Women's Health 2006, 2: pp. 761-772; The Oral Contraceptive and Hemostasis Study Group, The Effects of Seven Monophasic Oral Contraceptive Regimens on Hemostatic Variables: Conclusions From A Large Randomised Multicenter Study, Contraception 2003; 67: pp. 173-85; Rosendaal F R, Helmerhorst F M, Vandenbroucke J P, Oral Contraceptives, Hormone Replacement Therapy and Thrombosis, Thromb. Haemost. 2001, 86: pp. 112-23).

Several observational studies have also suggested that OCs containing the so-called third generation progestogens may be associated with a higher risk than OCs containing levonorgestrel (LNG) (Helmerhorst F M, Rosendaal F R, Vandenbroucke J P, ibid; Gomes M P V, Deitcher S R, ibid; Tchaikovski S, Tan G, Rosing J, ibid; Jick H, Kaye J A, Vasilakis-Scaramozza C, Jick S S, Risk of Venous Thromboembolism Among Users of Third Generation Oral Contraceptives Compared with Users of Oral Contraceptives with Levonorgestrel before and after 1995: Cohort and Case-control Analysis, BMJ 2000, 321: pp. 1190-1195; and Bloemenkamp K W M, Epidemiology of Oral Contraceptive Related Thrombosis, Thromb. Res. 2005, 1155: pp. 1-6).

However, in young women DVT is a rare event and, consequently, fairly reliable epidemiological findings cannot be expected before new formulations have been used by a large number of women for a sufficient period of time. As a surrogate, regulatory authorities demand controlled studies on the effect of new OC formulations or regimens on hemostatic parameters, even though no causal relationship has so far been established between OC-induced changes in distinct hemostatic parameters and the risk of venous thromboembolic disease.

Most of the hemostatic parameters are produced in the liver and may be modified by OCs. This is influenced primarily by EE, whereas progestogens may partly attenuate the estrogenic effect. Therefore, the effects on many hemostatic variables are dependent on the dose of EE, while the various progestogens may differ with respect to their modifying action on estrogen-dependent hemostatic parameters (Kuhl H, Effects of Progestins on Haemostasis, Maturitas 1996, 24: pp. 1-19). Progestogens are not a homogeneous class of hormones, but differ in their pattern of hormonal activity. Concerning their effect on coagulation and fibrinolysis, progestogens with androgenic properties like LNG may counteract the effect of EE on some hemostatic factors, whereas progestogens without androgenic activity, e.g., chlormadinone acetate, cyproterone acetate (CPA) or drospirenone (DRSP), have no influence. Dienogest (DNG) is the only nortestosterone derivative with no androgenic, but antiandrogenic activity and, therefore, no expected effects on EE-induced changes in hemostasis (Wiegratz I, Lee J H, Kutschera E, Winkler U H, Kuhl H, Effect of Four Oral Contraceptives on Hemostatic Parameters, Contraception 2004, 70: pp. 97-106). Irrespective of their effect on hemostasis, EE and the various progestogens may directly affect the function of the endothelium and smooth muscle cells. This is exemplified by certain progestogens like medroxyprogesterone acetate, gestodene (GSD) or 3-keto-desogestrel which may upregulate the thrombin receptor and tissue-factor and, hence, the pro-coagulatory activity in the vessel wall, probably owing to their intrinsic glucocortioid activity (Herkert O, Kuhl H, Sandow J, Busse R, Schini-Kerth V B, Sex steroids Used in Hormonal Treatment Increase Vascular Procoagulant Activity by Inducing Thrombin Receptor (PAR-1) Expression, Role of the Glucocorticoid Receptor, Circulation 2001, 104: pp. 2826-31).

### BRIEF SUMMARY OF THE INVENTION

It is an object of the present invention to provide a monophasic pharmaceutical preparation with contraceptive action without treatment of thrombosis or hemostatic parameters.

It is a further object of the present invention to provide an oral contraceptive preparation for long-cycle administration, which does not have a negative effect on hemostatic variables and blood pressure.

It is another object of the present invention to provide a kit for contraception that comprises daily dose units of the contraceptive preparation according to the present invention, which does not have negative effects on hemostatic variables and blood pressure, especially during long-cycle oral administration.

It is an additional object of the present invention to provide a kit for contraception that contains 91 daily dose units, which include 84 daily dose units containing a combination of dienogest and ethinyl estradiol and 7 daily dose units that do not contain any active contraceptive agent or contain a placebo.

It is an additional object of the present invention to provide a method of treating a woman to prevent pregnancy or conception using the kit and/or contraceptive preparation according to the present invention.

These objects and others, which will be made more apparent hereinafter, are attained by a monophasic contraceptive preparation for long-cycle administration without affecting blood pressure, which contains a daily dose unit comprising a combination of 2.0 mg of dienogest and 0.030 mg of ethinyl estradiol.

In a preferred embodiment of the invention the contraceptive preparation is in the form of an orally administered tablet containing 2.0 mg of dienogest and 0.030 mg of ethinyl estradiol.

The kit for long-cycle administration of the monophasic contraceptive preparation according to the invention comprises at least n×21 daily dose units of the contraceptive preparation, each of which contains 2.0 mg of dienogest and 0.030 mg of ethinyl estradiol, with n=2, 3, 4, 5, 6, 7, 8, 9, 10, 11, 12, 13, 14, 15, 16, and 17, and at most 7 daily dose units, each of which contains no active contraceptive agent or a placebo.

An especially preferred embodiment of the kit for a 91-day cycle contains 84 daily dose units of the contraceptive preparation of the present invention and 7 daily dose units containing a placebo or no active contraceptive agent, wherein each of the 84 daily dose units of the contraceptive preparation contains a combination of 2.0 mg of dienogest and 0.030 mg of ethinyl estradiol.

A process for making the monophasic contraceptive preparation for long-cycle administration is also part of the present invention. This process includes making a plurality of daily dose units, each of the daily dose units containing a combination of 2.0 mg of dienogest and 0.030 mg of ethinyl estradiol.

A method of treating a woman in order to prevent pregnancy or conception without affecting blood pressure is another aspect of the present invention. This method comprises the steps of:

a) administering to the woman daily a combination of 2.0 mg of dienogest and 0.030 mg of ethinyl estradiol for at least n×21 days, wherein n=2, 3, 4, 5, 6, 7, 8, 9, 10, 11, 12, 13, 14, 15,16, and 17; and

b) after the administering of the combination for at least n×21 days, administering daily to the woman a placebo or no active contraceptive agent for at most 7 days; and then c) repeating steps a) and b) for a time period during which pregnancy or conception is to be prevented.

In a preferred embodiment of the method of treatment the combination of 2.0 mg of dienogest and 0.030 mg of ethinyl estradiol is administered daily to the woman for 84 days and thereafter the placebo or no active contraceptive agent is administered daily to the woman for 7 days so that the cycle length is 91 days.

By repeating the 91-day cycle four times effective contraceptive action is provided for an entire year.

More generally depending on the desire of the women for continued contraceptive action without affecting blood pressure combined with a need for freedom from bleeding over the entire administration time interval the total number of successively administered daily dose units containing the combination of dienogest and ethinyl estradiol in the kit can be as much as 84 and the number of daily dosage units containing no active contraceptive agent or containing a placebo can be 7, so that the total number of cycle days is n×21 plus 7), n being equal to 2, 3, or 4.

It was unexpected that a long-cycle oral contraceptive preparation could be formulated that does not adversely affect blood pressure, which contains ethinyl estradiol and dienogest, a gestagen that is not an aldosterone antagonist. It was also unexpected that long-cycle administration of this pharmaceutical preparation would not cause any significantly greater changes in hemostatic variables than conventional administration of this same preparation using a 28-day cycle, in which the same contraceptive preparation was administered for 21 days and no contraceptive agent or a placebo was administered for 7 days of the conventional cycle.

### BRIEF DESCRIPTION OF THE DRAWING

The objects, features and advantages of the invention will now be illustrated in more detail with the aid of the following description of the experimental results, with reference to the accompanying figures in which:

FIG. 1 is a diagrammatic comparison of a conventional treatment and long-cycle treatment with the oral contraceptive preparation according to the invention over the course of a year, which also shows blood sampling scheduled during the control cycle and at 3 months and 12 months of treatment;

FIGS. 2a to 2d are graphical illustrations showing the effects of conventional and long-cycle administration of the combination of EE and DNG according to the present invention on the plasma concentration of fibrinogen, of Factor VII antigen, and on the activity of Factor VII and Factor VIII (mean±S.D., p<0.05 vs. control cycle, p<0.01 vs. control cycle, and p<0.01 vs. 3^{rd} month);

FIGS. 3a to 3d are graphical illustrations showing the effects of conventional and long-cycle administration of the combination of EE and DNG according to the present invention on the activity of antithrombin and of protein C and on the plasma concentration of free protein S and the thrombin-antithrombin complex (mean±S.D., p<0.05 vs. control cycle, p<0.01 vs. control cycle, and p<0.05 vs. 3^{rd} month); and

FIGS. 4a to 4d are graphical illustrations showing the effects of conventional and long-cycle administration of the combination of EE and DNG according to the present invention on the plasma concentration of plasminogen, plasminogen-activator-inhibitor-1 antigen, and plasmin-antiplasmin complex, and on the activity of the tissue-plasminogen activator (mean±S.D., p<0.05 vs. control cycle, p<0.01 vs. control cycle, and p<0.05 vs. 3rd month).

### COMPARATIVE EXPERIMENTAL RESULTS

1. Basic Experimental Procedure

In a comparative, prospective, randomized study at the Center of Obstetrics and Gynecology, University Hospital of Frankfurt, the effect of conventional treatment (21+7 days) with a monophasic combination of 30 μg EE and 2 mg DNG (EE/DNG) on various procoagulatory, anticoagulatory, profibrinolytic factors, antifibrinolytic factors, markers of thrombin and fibrin turnover, and clotting tests was compared with that of a long-cycle regimen (84+7 days) with EE/DNG. The time-points were 3 months and 12 months.

2. Materials and Methods

2.1 Design of the Study

After screening of 66 healthy women between 20 and 33 years of age seeking contraception, this experimental randomized study included sixty (60) volunteers with regular menstrual cycles and without contraindications for the use of OCs. These women had not used any hormonal medication for at least 4 weeks prior to the study and did not use drugs that were known to influence the effects of OCs.

A general and gynecological examination including a cervical cytology, a pregnancy test, and an assessment of the general safety laboratory parameters were performed before treatment, during treatment (3 months), and at the end of treatment (12 months).

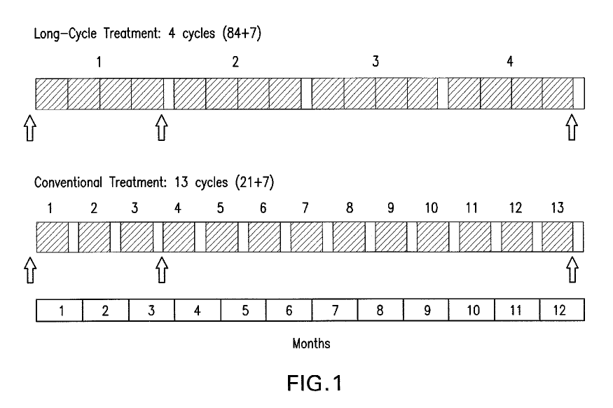

After the control cycle, the volunteers were randomly assigned to take EE/DNG either conventionally (13 cycles with 21 days of treatment+7 days without hormones) (n=29) or according to a long-cycle regimen (4 extended cycles with 84 days of continuous administration+7 days without hormones) (n=30), as shown in FIG. 1.

Blood was taken on days 21-26 of the control cycle and on days 82-84 of the first and fourth long-cycle (months 3 and 12, respectively); or on days 19-21 of the third and thirteenth conventional cycle (months 3 and 12, respectively). Blood sampling was carried out in the morning (08:00-10:00 a.m.) after overnight fasting in a lying position.

Each volunteer kept a cycle diary in which the intake of the tablets and the bleeding events were recorded daily. Within 28 days after termination of treatment a final examination was performed (general and gynecological examination including cervical cytology, safety laboratory tests and pregnancy test). At the visits, the volunteers were to report the occurrence of any adverse events.

The study was approved by the Ethics Committee of the University Hospital Frankfurt and was conducted in compliance with Good Clinical Practice (GCP) and the Declaration of Helsinki. Before enrollment in the study, a written informed consent was obtained from each volunteer.

2.2 Laboratory Methods

For the analyses of hemostatic variables, the blood samples were centrifuged and the citrate plasma was stored at −70° C. until analysis. The plasma parameters were determined by the laboratory LKF GmbH, Kiel, Germany.

Procoagulatory Variables

Fibrinogen was measured coagulometrically by the method of Claus using a kit from Roche Diagnostics GmbH (Mannheim, Germany), Factor VII activity (VIIc) by a clotting test (one-stage method) using factor VII deficient plasma from Dade Behring (Marburg, Germany), activated Factor VII (VIIa) by a clotting test (STACLOT® VIIa-rTF) from Roche Diagnostics GmbH (Mannheim, Germany), Factor VII antigen by a commercial enzyme-linked immunosorbent assay (ELISA, ASSERACHROM® VII:Ag) from Roche Diagnostics GmbH (Mannheim, Germany), and Factor VIII activity (VIIIc) by a clotting test (one-stage method) using factor VII deficient plasma from Dade Behring (Marburg, Germany).

Anticoagulatory Variables

Antithrombin antigen was determined nephelometrically by N Antiserum to Antithrombin III from Dade Behring (Marburg, Germany), Antithrombin activity using chromogenic substrate (COAMATIC® LR Antithrombin) from Chromogenix (Milano, Italy), Protein C antigen by an ELISA (REAADS® Protein C Antigen Testkit) from Corgenic Inc. (Westminster, Colo., USA), Protein C activity using chromogenic substrate (COAMATIC® Protein C) from Chromogenix, Free Protein S and Total Protein S by an ELISA (REAADS® Protein S Antigen Test kit) from Corgenix Inc.

Profibrinolytic Variables

Plasminogen was measured using chromogenic substrate (COAMATIC® Plasminogen) from Chromogenix, Tissue plasminogen activator (t-PA) antigen by an ELISA (t-PA Antigen ELISA) from Technoclone GmbH (Vienna, Austria), and t-PA activity by an ELISA (t-PA ACTIBIND®) Technoclone GmbH (Vienna, Austria).

Antifibrinolytic Variables

Plasminogen activator inhibitor-1 (PAI-1) antigen was measured by an ELISA (PAI-1 ACTIBIND®) from Technoclone GmbH.

Markers of Thrombin and Fibrin Formation and Turnover

Thrombin-antithrombin complex (TAT) was measured by an ELISA (Enzygnost TAT micro) from Dade Behring, Plasmin-α2-antiplasmin complex (PAP) by an ELISA (PAP micro) from DRG Instruments GmbH (Marburg, Germany), Prothrombin fragments 1+2 by an ELISA (Enzygnost F 1+2 micro) from Dade Behring, D-Dimers by an ELISA (Dimertest gold) from Agen Biomedical LTD (Acacia Ridge, Australia).

Clotting Tests

Prothrombin time (PT) (Quick test) and Activated partial thromboplastin time (APTT) were determined by clotting tests (THROMBOREL® S and Pathromtin SL) from Dade Behring.

2.3 Statistical Analysis

The sample size was established as 60 subjects, i.e., 30 in each study arm. This sample size is commonly used to describe hemostasis changes under hormonal contraceptives. All variables were analyzed on the basis of the full analysis set (FAS). A volunteer was included in the FAS if she had taken at least one tablet of the study medication and if at least one observation after dosing was available. The study variables were descriptively evaluated by comparing the means at each examination time and their changes over time. Missing data were not replaced. The changes over time (i.e., the absolute changes for within-group comparisons between time-points) were evaluated by means of the Wilcoxon signed rank-test. Comparisons between groups were carried out using the Wilcoxon rank sum test. The significance level was p=0.05.

3. Results

3.1. Disposition of Volunteers

Sixty volunteers were randomized to be treated with EE/DNG either for 13 conventional cycles (21+7 days) or 4 long-cycles (84+7 days). One volunteer randomized to the conventional regimen became pregnant in the control cycle and dropped out before the first medication, reducing this group to n=29. Two women discontinued the study prematurely during the first long-cycle because of irregular bleeding and tinnitus, respectively. Consequently, 57 subjects completed the study (conventional n=29, long-cycle n=28).

Statistical analysis was carried out on the basis of full analysis set (FAS) (n=59).

Both treatment groups were comparable in terms of baseline data for age and mean body mass index (Table I). The latter did not substantially change during treatment. No relevant change in mean blood pressure or pulse rate was observed during the study.

TABLE I
CHARACTERISTICS OF STUDY GROUPS AT SCREENING

| | Conventional | Long-cycle |
|---|---|---|
| | Regimen | Regimen |
| Characteristic* | (FAS, n = 29) | (FAS, n = 30) |
| Age (years) | 24.6 ± 3.2 | 25.1 ± 2.6 |
| Weight (kg) | 63.7 ± 8.0 | 62.0 ± 8.1 |
| BMI (kg/m²) | 22.3 ± 2.8 | 21.8 ± 2.6 |
| Systolic Blood Pressure | 109.1 ± 10.9 | 112.2 ± 10.6 |
| | mm Hg | mm Hg |
| Diastolic Blood Pressure | 71.0 ± 7.2 | 69.8 ± 9.1 |
| | mm Hg | mm Hg |
| Heart Rate (beats/min) | 71.9 ± 7.6 | 70.9 ± 7.9 |

*Characteristic values expressed as a mean ± SD.

3.2. Procoagulatory Factors

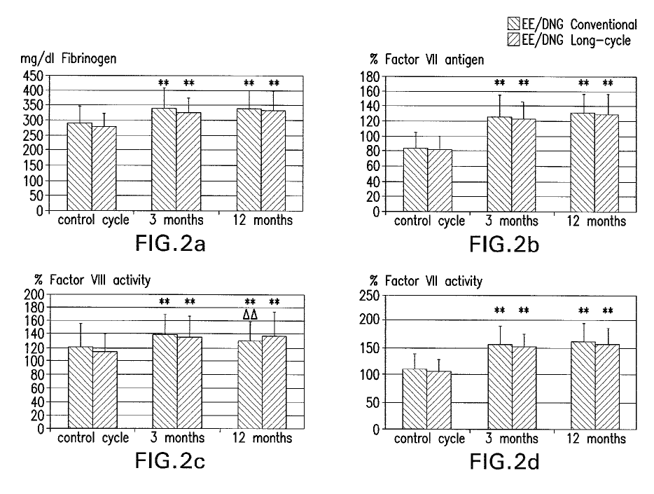

There was no significant difference between both regimens in the level or activity of procoagulatory factors at any given time. Fibrinogen was significantly elevated by approximately 20% in both groups in the 3^{rd} and 12^{th} month (FIG. 2a). Both during conventional and long-cycle treatment there was a 30-60% rise in Factor VII antigen, FVII activity and activated FVII. The rise observed in the 3^{rd} month was only slightly enhanced until the 12^{th} month of treatment (FIGS. 2b, 2d, Table II herein below). Concerning Factor VIII activity, an increase of 15-20% was measured in the 3^{rd} month in both groups. Thereafter, the activity tended to decrease during conventional use, but did not change in the long-cycle group (FIG. 2c).

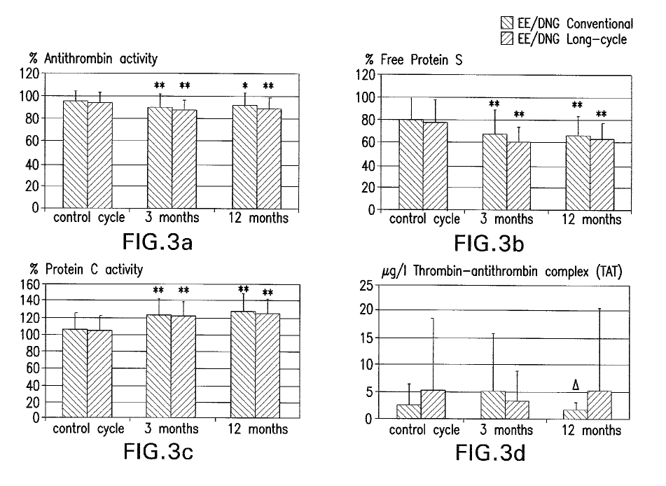

Conventional use of EE/DNG caused a slight increase in the level of prothrombin fragment 1+2 and a moderate increase during the extended cycle regimen, but the difference between both regimens was not significant (Table II). The plasma concentration of TAT showed large inter-individual variations in the control cycle and during treatment with EE/DNG in both groups. However, the changes from the control cycle were not significant (FIG. 3d).

3.3. Anticoagulatory Factors

In both groups, a marginal decrease in the level and activity of antithrombin was observed at the 3^{rd} and 12^{th} months of treatment (FIG. 3a). Free protein S and total protein S were reduced by about 20% (FIG. 3b, Table II). In contrast, protein C antigen and activity rose by 15-20% at the 3^{rd} and 12^{th} months of treatment in both regimens (FIG. 3c, Table II).

3.4. Variables of Fibrinolysis

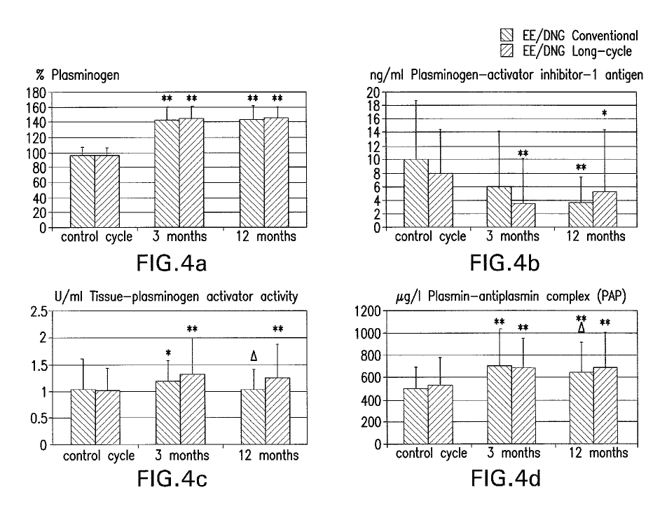

In the 3^{rd} month of treatment, there was a comparable rise in the plasminogen level (about 50%) in both groups. This did not change in the 12^{th} month (FIG. 4a). The t-PA antigen was reduced by 30% both under conventional and long-cycle regimens In the conventional group, the t-PA activity was elevated by 15% only in the 3^{rd} month, whereas in the long-cycle group it increased by 30% and 25% in the 3^{rd} and 12^{th} month of treatment, respectively (Table II, FIG. 4c). There was a pronounced increase in the two activation markers of fibrinolyis, PAP and D-dimers, by 30-40% and 20-55%, respectively, but owing to the large inter-individual variations no significant difference between the regimens was found (Table II and FIG. 4d). In both groups, the level of PAI-1 decreased by 40-50% in the 3^{rd} month and by 35-60% in the 12^{th} month (FIG. 4b).

3.5. Global Clotting Tests

Both clotting tests were only slightly influenced; while the prothrombin time increased by about 10%, the APTT showed a 10% acceleration (Table II).

The resulting average values of the hemostatic variables for conventional administration and long-cycle administration of the oral contraceptive preparation of the present invention are presented below in Table II along with statistical measures of the precision of the results.

TABLE II
EFFECTS OF CONVENTIONAL AND LONG-CYCLE ADMINISTRATION
OF A MONOPHASIC COMBINATION OF 30 μG ETHINYL ESTRADIOL AND 2 mg
DIENOGEST (EE/DNG) ON VARIOUS HEMOSTATIC VARIABLES AFTER 3 AND
12 MONTHS OF TREATMENT COMPARED TO THE CONTROL CYCLE

| | EE/DNG Conventional | | | EE/DNG - LONG-CYCLE | | |
|---|---|---|---|---|---|---|
| Parameter, as | Control | 3 months | 12 months | Control | 3 months | 12 months |
| mean ± S.D. | (n = 29) | (n = 29) | (n = 29) | (n = 30) | (n = 30) | (n = 30) |
| Activated Factor VII | 97 ± 47 | 128 ± 47** | 143 ± 62** | 103 ± 43* | 140 ± 61** | 148 ± 69** |
| (FVIIa), mU/mL | | | | | | |
| Prothrombin fragment | 0.87 ± 0.32 | 1.01 ± 0.57 | 0.91 ± 0.30# | 0.84 ± 0.43 | 1.00 ± 0.43** | 1.07 ± 0.71** |
| 1 + 2, nmol/L | | | | | | |
| Antithrombin antigen, | 240 ± 26 | 231 ± 26** | 232 ± 27** | 239 ± 22 | 224 ± 20** | 230 ± 23* |
| mg/L | | | | | | |
| Protein C antigen, % | 88 ± 17 | 101 ± 18** | 103 ± 19** | 87 ± 16 | 100 ± 14** | 103 ± 14** |
| Total protein S, % | 72 ± 14 | 58 ± 12** | 58 ± 13** | 73 ± 13 | 55 ± 10** | 55 ± 10** |
| t-PA antigen, ng/mL | 1.4 ± 0.7 | 0.9 ± 0.3** | 1.0 ± 0.6** | 1.6 ± 1.2 | 1.1 ± 1.1** | 1.2 ± 1.1** |
| D-dimers, ng/mL | 189 ± 139 | 267 ± 174** | 228 ± 115* | 198 ± 222 | 307 ± 372** | 255 ± 210* |
| Prothrombin time, % | 98 ± 9 | 109 ± 9** | 109 ± 11** | 99 ± 10 | 107 ± 8** | 108 ± 11** |
| Activated Partial | 33 ± 3 | 29 ± 2** | 29 ± 2** | 32 ± 3 | 29 ± 2** | 29 ± 3** |
| Thromboplastin time, | | | | | | |
| sec | | | | | | |

*p < 0.05 vs. control cycle;
**p < 0.01 vs. control cycle; and
#(n = 27) due to two implausible values

4. Discussion of Results and Comparison to Prior Art

The above-described study is the first randomized study comparing directly the effect of conventional and long-cycle administration of an OC on various hemostatic parameters. The most important finding was the lack of any significant differences between the conventional (21+7 days) and long-cycle regimen (84+7 days) at 12 months. In both groups there was a significant rise in fibrinogen, factor VII and factor VIII, protein C, plasminogen, and t-PA activity and a reduction in antithrombin, protein S, t-PA antigen, and PAI-1 antigen. In parallel the timeover means of prothrombin fragment 1+2, D-dimers, and PAP increased.

There are two other studies on the effect of extended OC regimens on hemostasis. Long-cycle treatment (63+7 days) of 20 women or conventional treatment (21+7 days) of 10 women with an OC containing 30 μg EE+150 μg desogestrel (DG) for 12 months resulted in similar changes in various hemostatic parameters. After 3 months of treatment with the long-cycle regimen fibrinogen was increased by 10%, factor VII by 30% and TAT complex by 175%, whereas t-PA antigen was reduced by 45% and PAI activity by 90%; antithrombin, protein C, and protein S were not significantly changed (Cachrimanidou A C, Hellberg D, Nilsson S, von Schoultz B, Crona N, Siegbahn A, Hemostasis Profile and Lipid Metabolism with Long-interval Use of a Desogestrel-containing Oral Contraceptive, Contraception 1994, 50: pp. 153-65).

In a non-comparative study with 45 women on the continuous use of 30 μg EE and 75 μg GSD for 24 weeks the levels of fibrinogen (+10%) and PAI-1 (+45%) increased and the prothrombin time decreased, whereas antithrombin, protein C, protein S, and the activated partial thromboplastin time were not significantly altered (Bonassi Machado R, Fabrini P, Machado Cruz A, Maia E, da Cunha Bastos A, Clinical and Metabolic Aspects of the Continuous Use of a Contraceptive Association of Ethinyl estradiol (30 μg) and Gestodene (75 μg), Contraception 2004, 70: pp. 365-70).

The present study confirmed previous results on estrogen-induced changes in various hemostatic variables during 1 year of treatment with EE/DNG. This OC can be regarded as an estrogen-predominant formulation concerning its effect on certain hormone-sensitive hepatic proteins. A study with preparations containing 2 mg DNG and different estrogen components has revealed that the changes of several hemostatic variables were dependent on the dose of EE (Wiegratz I, Lee J H, Kutschera E, Winkler U H, Kuhl H, Effect of Four Oral Contraceptives on Hemostatic Parameters, Contraception 2004, 70: pp. 97-106). As DNG has no androgenic, but even antiandrogenic activities, it does not attenuate the estrogen-induced alterations in the hepatic production of hormone-sensitive proteins including hemostatic factors (The Oral Contraceptive and Hemostasis Study Group, The Effects of Seven Monophasic Oral Contraceptive Regimens On Hemostatic Variables: Conclusions From A Large Randomised Multicenter Study, Contraception 2003, 67: pp. 173-85; Kuhl H, Effects of Progestins on Haemostasis, Maturitas 1996, 24: pp. 1-19; Winkler U H, Effects on Hemostatic Variables of Desogestrel- and Gestodene-containing Oral Contraceptives in Comparison with Levonorgestrel-containing Oral Contraceptives: a Review, Am. J. Obstet. Gynecol. 1998, 179: pp. S51-S61; Norris L A, Bonnar J, The Effect of Oestrogen Dose and Progestin Type on Hemostatic Changes in Women Taking Low-dose Oral Contraceptives, Br. J. Obstet. Gynaecol. 1996, 103: pp. 261-7).

The increase in both procoagulatory and fibrinolytic activity observed during treatment with most OCs suggests a shift of the equilibrium between coagulation and fibrinolysis to a higher level of fibrin turnover. These changes reflect an effect of the EE component particularly on fibrinogen, factor VII, antithrombin, protein S, plasminogen, and t-PA leading to an enhanced formation of prothrombin fragments 1+2 and D-dimers as well as TAT and PAP (Spona J, Feichtinger W, Kindermann C, Schneider B, Mellinger U, Walter F, Moore C, Gräser T, Double-blind, Randomized, Placebo-controlled Study on the Effects of the Monophasic Oral Contraceptive Containing 30 μg Ethinyl estradiol and 2 mg Dienogest on the Hemostatic System, Contraception 1997, 56: pp. 67-75; Task Force on Oral contraceptives-WHO Special Programme of Research. A Multicentre Study of Coagulation and Hemostatic Variables during Oral Contraception: Variations with Four Formulations, Br. J. Obstet. Gynaecol. 1991, 98: pp. 1117-28 ; Winkler U H, Oberhoff C, Bier U, Schindler E A, Gillain D, Hemostatic Effects of Two Oral Contraceptives Containing Low Doses of Ethinyl Estradiol and either Gestodene or Norgestimate: an Open, Randomised, Parallel-group Study, Int. J. Fertil. 1995, 40: pp. 260-8; Tans G, Curvers J, Middledorp S, Thomassen M C, Meijers J C, Prins M H, Bouma B N, Büller H R, Rosing J, A Randomised Cross-over Study on the Effects of Levonorgestrel- and Desogestrel-containing Oral Contraceptives on the Anticoagulant Pathways, Thromb. Haemost. 2000, 84: pp. 15-21; Maim J Laurel M, Dahlbäck B, Changes in the Plasma Levels of Vitamin K-dependent Protein C and S and of C4b-binding Protein during Pregnancy and Oral Contraception, Br. J. Haematol. 1988, 68: pp. 437-43). Several studies have revealed that these changes are less pronounced under LNG-containing OCs than with OCs containing a third generation progestogen (The Oral Contraceptive and Hemostasis Study Group, The Effects of Seven Monophasic Oral Contraceptive Regimens On Hemostatic Variables: Conclusions From A Large Randomised Multicenter Study, Contraception 2003, 67: pp. 173-85 (The Oral Contraceptive and Hemostasis Study Group, The Effects of Seven Monophasic Oral Contraceptive Regimens On Hemostatic Variables: Conclusions From A Large Randomised Multicenter Study, Contraception 2003, 67: pp. 173-85; Kemmeren J M, Algra A, Meijers J C M, Bouma B N, Grobbee D E, Effects of Second and Third generation Oral Contraceptives and Their Respective Progestagens on the Coagulation System in the Absence or Presence of the Factor V Leiden Mutation, Thromb Haemost. 2002, 87: pp. 199-205; Kluft C, Effects on Hemostasis Variables by Second and Third Generation Combined Oral Cotraceptives: A Review of Directly Comparative Studies, Curr. Med. Chem. 2000, 7, pp. 585-91; Bloemenkamp K W M, Rosendaal F R, Helmerhorst F M, Koster T, Berina R M, Vandenbroucke J P, Hemostatic Effects of Oral Contraceptives in Women Who Developed Deep-vein Thrombosis while Using Oral Contraceptives, Thromb. Haemost. 1998, 80: pp. 382-7; Tans G, Bouma B N, Büller H R, Rosing J, Changes of Hemostatic Variables During Oral Contraceptive Use, Semin. Vasc. Med. 2003, 3: pp. 61-68).

The rise in fibrinogen and FVII activity observed in a previous study (Wiegratz I, Lee J H, Kutschera E, Winkler U H, Kuhl H, ibid) during the first three cycles with EE/DNG corresponds to that in the present study which also showed a close correlation between FVII concentration and FVII activity.

The effects on coagulation inhibitors, i.e., a slight decrease in antithrombin, a moderate rise in protein C and a moderate decrease in protein S, are similar to that observed in a previous study with EE/DNG (Wiegratz I, Lee J H, Kutschera E, Winkler U H, Kuhl H, ibid), and agree with those in women treated with various other low-dose OCs (The Oral Contraceptive and Hemostasis Study Group, The Effects of Seven Monophasic Oral Contraceptive Regimens On Hemostatic Variables: Conclusions From A Large Randomised Multicenter Study, Contraception 2003, 67: pp. 173-85; Spona J, Feichtinger W, Kindermann C, Schneider B, Mellinger U, Walter F, Moore C, Gräser T, Double-blind, Randomized, Placebo-controlled Study on the Effects of the Monophasic Oral Contraceptive Containing 30 μg Ethinyl estradiol and 2 mg Dienogest on the Hemostatic System, Contraception 1997, 56: pp. 67-75; Task Force on Oral contraceptives-WHO Special Programme of Research, A Multicentre Study of Coagulation and Hemostatic Variables during Oral Contraception: Variations with Four Formulations, Br. J. Obstet. Gynaecol. 1991, 98: pp. 1117-28; Winkler U H, Oberhoff C, Bier U, Schindler E A, Gillain D, Hemostatic Effects of Two Oral Contraceptives Containing Low Doses of Ethinyl Estradiol and either Gestodene or Norgestimate: an Open, Randomised, Parallel-group Study, Int. J. Fertil. 1995, 40: pp. 260-8; Tans G, Curvers J, Middledorp S, Thomassen M C, Meijers J C, Prins M H, Bouma B N, Büller H R, Rosing J, A Randomised Cross-over Study on the Effects of Levonorgestrel- and Desogestrel-containing Oral Contraceptives on the Anticoagulant Pathways, Thromb. Haemost. 2000, 84: pp. 15-21; Malm J Laurel M, Dahlbäck B, Changes in the Plasma Levels of Vitamin K-dependent Protein C and S and of C4b-binding Protein during Pregnancy and Oral Contraception, Br. J. Haematol. 1988, 68: pp. 437-43; Kemmeren J M, Algra A, Meijers J C M, Bouma B N, Grobbee D E, Effects of Second and Third generation Oral Contraceptives and Their Respective Progestagens on the Coagulation System in the Absence or Presence of the Factor V Leiden Mutation, Thromb Haemost. 2002, 87: pp. 199-205; and Granata A, Sobbrio G A, D'Arrigo F, Bacillari M, De Luca P, Egitto M, Granese D, Pulle C, Trimarchi F, Changes in the Plasma Levels of Protein C and S in Young Women on Low-dose Oestrogen Oral Contraceptives, Clin. Exp. Obstet. Gynecol. 1991, 18: pp. 9-12).

Similar to the results of a previous study with EE/DNG, the changes in plasminogen, t-PA activity and PAI-1 suggest an activation of the fibrinolytic system as reflected by a rise in the levels of PAP and D-dimers (Wiegratz I, Lee J H, Kutschera E, Winkler U H, Kuhl H, ibid).

Altogether, the changes in the various hemostatic variables observed during treatment with EE/DNG correspond to those reported for other OCs. The moderate shortening of the prothrombin time and APTT may be related to the increase in the activity of FVII and FVIII; however, the in vivo activation of protein C which may attenuate coagulation, does not take place during the in vitro clotting test.

Although OCs may affect most hemostatic factors, a plausible biological explanation for the increased risk of thromboembolic disease is lacking, because some of the changes are prothrombotic and others can be regarded as antithrombotic (Tans G, Bouma B N, Büller H R, Rosing J, Changes of Hemostatic Variables During Oral Contraceptive Use, Semin. Vasc. Med. 2003, 3: pp. 61-68). Some findings, however, may disclose some relationship between hemostasis peculiarities and elevated thrombosis risk. The rise in FVII and FVIII and the reduction in antithrombin and protein S observed during OC use was more pronounced in women who developed DVT during treatment with OCs (Bloemenkamp K W M, Rosendaal F R, Helmerhorst F M, Koster T, Berina R M, Vandenbroucke J P, Hemostatic Effects of Oral Contraceptives in Women Who Developed Deep-vein Thrombosis while Using Oral Contraceptives, Thromb. Haemost. 1998, 80: pp. 382-7). Tissue-factor-initiated thrombin generation is strongly increased by OC use in women with prior thrombosis suggesting prothrombotic abnormalities (Brummel-Ziedins K E, Vossen C Y, Butenas S, Mann K G, Rosendaal F R, Thrombin Generation Profiles in Deep Venous Thrombosis, J. Thromb. Haemost. 2005, 3: pp. 2497-505). The rapid development of an acquired APC resistance during administration of estrogen-predominant OCs (which is rapidly reversed after stopping intake), is probably associated with the reduction in free protein S (Tans G, Bouma B N, Büller H R, Rosing J, Changes of Hemostatic Variables During Oral Contraceptive Use, Semin. Vasc. Med. 2003, 3: pp. 61-68).

It has been suggested that the activation of procoagulatory factors by OCs may be outbalanced by an elevated fibrinolytic activity. However, recent findings indicate that the increased fibrinolytic potential may be counteracted by an elevated resistance to clot-lysis caused by the thrombin-activatable fibrinolytic inhibitor (TAFI) which is increased by OCs, particularly those containing third generation progestogens (Tans G, Curvers J, Middledorp S, Thomassen M C, Meijers J C, Prins M H, Bouma B N, Büller H R, Rosing J, A Randomised Cross-over Study on the Effects of Levonorgestrel- and Desogestrel-containing Oral Contraceptives on the Anticoagulant Pathways, Thromb. Haemost. 2000, 84: pp. 15-21 and Tans G, Bouma B N, Büller H R, Rosing J, Changes of Hemostatic Variables During Oral Contraceptive Use, Semin. Vasc. Med. 2003, 3: pp. 61-68).

The question remains whether the lower risk of DVT in women using LNG-containing OCs as compared to OCs containing GSD, DG, or CPA is causally related to the more marked estrogen-induced changes in hemostatic variables (Gomes M P V, Deitcher S R, Risk Of Venous Thromboembolic Disease Associated With Hormonal Contraceptives And Hormone Replacement Therapy, Arch. Intern. Med. 2004, 164: pp. 1965-1976; Tchaikovski S, Tan G, Rosing J, Venous Thrombosis And Oral Contraceptives: Current Status, Women's Health 2006, 2: pp. 761-772; Jick H, Kaye J A, Vasilakis-Scaramozza C, Jick S S, Risk of Venous Thromboembolism Among Users of Third Generation Oral Contraceptives Compared with Users of Oral Contraceptives with Levonorgestrel before and after 1995: Cohort and Case-control Analysis, BMJ 2000, 321: pp. 1190-1195; and Bloemenkamp K W M, Epidemiology of Oral Contraceptive Related Thrombosis, Thromb. Res. 2005, 1155: pp. 1-6; and Seaman H E, de Vries C S, Farmer R D T, The Risk of Venous Thromboembolism in Women Prescribed Cyproterone Acetate in Combination with Ethinyl Estradiol: a Nested Cohort Analysis and Case-control Study, Hum. Reprod. 2003, 18: pp. 522-6).

It has been claimed that the “estrogenicity” of the formulation as reflected by the rise in the hepatic production of sex hormone-binding globulin (SHBG) might be a measure for the risk of venous thromboembolic disease. However, recent observational studies revealed that the use of OCs containing DNG, norgestimate (NGM) or DRSP (which may also be regarded as estrogen-predominant formulations), is not associated with a higher risk of DVT as compared to LNG-containing OCs (Jick S S, Kaye J A, Russmann S, Jick H, Risk of Nonfatal Thromboembolism with Oral Contraceptives Containing Norgestimate or Desogestrel Compared with Oral Contraceptives Containing Levonorgestrel, Contraception 2006, 73: pp. 566-70; Heinemann L A J, Moehner S, Assmann A, Heinemann K, Use of Oral Contraceptives Containing Dienogest and Risk of Venous Thromboembolism—an Extended Summary Report of a Case-control Study. Life and Medical Science Online 2001, 2: DOI 10.1072/LO211329; Dinger J C, Heinemann A J, Kühl-Habich D, The Safety of a Drospirenone-containing Oral Contraceptive: Final Results from the European Active Surveillance Study on Oral Contraceptives Based on 142, 475 Women-years of Observation, Contraception 2007, 75: pp. 344-54).

The present study compared the time-dependent changes in various hemostasis plasma parameters during conventional treatment with EE/DNG with those observed in women on a long-cycle regimen with EE/DNG. Until now, it remained unclear whether or not continuous treatment for 3 months with estrogen-dominant OCs causes a more pronounced rise in estrogen-dependent hepatic proteins than during the conventional regimen of 21+7 days.

It is well known that according to the rise in the serum levels of EE and the respective progestogen up to a steady-state during the first 10 days of conventional OC use, the change in hormone-dependent hepatic serum parameters follows this pattern and also approaches a steady-state (Jung-Hoffmann C, Kuhl H, Pharmacokinetics and Pharmacodynamics of Oral Contraceptive Steroids: Factors Influencing Steroid Metabolism, Am. J. Obstet. Gynecol. 1990, 163: pp. 2183-97; Wiegratz I, Jung-Hoffmann C, Kuhl H, Effect of Two Oral Contraceptives Containing Ethinyl Estradiol and Gestodene or Norgestimate Upon Androgen Parameters and Serum Binding Proteins, Contraception 1995, 51: pp. 341-6; and Jung-Hoffmann C, Heidt F, Kuhl H, Effect of Two Oral Contraceptives Containing 30 μg Ethinyl Estradiol and 75 μg Gestodene or 150 μg Desogestrel Upon Various Hormonal Parameters, Contraception 1988, 38: pp. 593-603).

In the first cycle of treatment with OCs containing EE and GSD or DG, the serum concentration of SHBG rises to a maximum on day 21 (Jung-Hoffmann C, Heidt F, Kuhl H, ibid). During the following hormone-free interval of 7 days, the effect is only partly reversed, but in the second treatment cycle SHBG rises again up to a peak level on day 11; this peak is higher than that in the first cycle, but does not differ significantly from that on day 21 (in other words the steady-state is reached earlier (Jung-Hoffmann C, Heidt F, Kuhl H, ibid). During the following cycles, SHBG reaches a steady-state on Day 11, which is similar to that observed in the second treatment cycle (Jung-Hoffmann C, Heidt F, Kuhl H, ibid). In another study, a steady-state in the serum concentrations of SHBG and corticosteroid-binding globulin (CBG) was also reached on day 11 of treatment with OCs containing EE and GSD or NGM, and the peak levels in cycle 3 were identical to those in cycle 6 and 12 (Wiegratz I, Jung-Hoffmann C, Kuhl H, Effect of Two Oral Contraceptives Containing Ethinyl Estradiol and Gestodene or Norgestimate Upon Androgen Parameters and Serum Binding Proteins, Contraception 1995, 51: pp. 341-6).

Accordingly, it can be expected that during a long-cycle regimen a steady-state of various hepatic serum parameters is also reached within the first 2 to 3 weeks of OC administration, even though it cannot be totally excluded that during continuous treatment the changes may be more pronounced than during conventional use of OCs. In a conventional OC cycle, the hepatic production of estrogen-sensitive hemostatic parameters increases (e.g. fibrinogen, factor VII) or decreases (e.g. antithrombin) during the first days of administration up to a steady-state, which is reversed during the following hormone-free interval. This pattern is repeated during all subsequent treatment cycles. As the half-lives of the various hemostatic variables are different, the conventional use of OCs might be characterized by unstable phases of the equilibrium between procoagulatory and fibrinolytic activity (Robinson G E, Burren T, Mackie I J, Bounds W, Walshe K, Faint R, Guillebaud J, Changes in Hemostasis After Stopping the Combined Contraceptive Pill, Implications for Major Surgery, BMJ 1991, 302: pp. 269-71). It remains to be elucidated whether a stable steady-state in hemostasis during a long-cycle regimen or the cyclic fluctuations of various hemostasis parameters during conventional use of OCs are associated with different risks of DVT.

This study clearly demonstrates that during treatment with EE/DNG there is no significant difference between the conventional and the long-cycle regimen with respect to any hemostatic parameters at any time. The study also revealed that the intermediate increase in the procoagulatory and fibrinolytic activity observed after 3 months of use did not substantially change during further treatment. Also the present study confirmed that a steady-state in the estrogen-dependent changes in hemostasis was reached already at 3 months consistently with a previous study (Wiegratz I, Lee J H, Kutschera E, Winkler U H, Kuhl H, Effect of Four Oral Contraceptives on Hemostatic Parameters, Contraception 2004; 70: pp. 97-106). Consequently, the results of the present study suggest that, as far as the EE/DNG-induced changes in hemostasis may be regarded as a marker of the risk of venous thromboembolic disease, there is no reason to expect a higher relative risk for the long-cycle regimen compared to the conventional use of EE/DNG. This, however, remains to be demonstrated by epidemiological data.

While the invention has been illustrated and described as embodied in a contraceptive preparation for long-cycle administration without affecting blood pressure, a kit for long-cycle administration of the contraceptive preparation, a method of making the contraceptive preparation, and a method of treating a woman with it to prevent pregnancy or conception, the invention is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A monophasic contraceptive preparation for long-cycle administration without affecting blood pressure, said contraceptive preparation comprising a daily dose unit, wherein said daily dose unit contains 2.0 mg of dienogest and 0.030 mg of ethinyl estradiol.

2. The contraceptive preparation as defined in claim 1, comprising an orally administered tablet, said tablet containing said daily dose unit.

3. A kit for long-cycle administration of a monophasic contraceptive preparation, said kit containing
at least n×21 daily dose units of the contraceptive preparation, wherein each of said at least n×21 daily dose units contains a combination of 2.0 mg of dienogest and 0.030 mg of ethinyl estradiol and n=2, 3, 4, 5, 6, 7, 8, 9, 10, 11, 12, 13, 14, 15, 16, and 17; and
at most 7 daily dose units, wherein each of said at most 7 daily dose units contains no active contraceptive agent or a placebo.

4. A kit for long-cycle administration of a monophasic contraceptive preparation, said kit containing 84 daily dose units of the contraceptive preparation and 7 daily dose units, said 7 daily dose units containing a placebo or no active contraceptive agent;
wherein each of said 84 daily dose units of the contraceptive preparation contains a combination of 2.0 mg of dienogest and 0.030 mg of ethinyl estradiol.

5. A process of preparing a monophasic contraceptive preparation for long-cycle administration, said process including the step of making a plurality of daily dose units, each of said daily dose units containing a combination of 2.0 mg of dienogest and 0.030 mg of ethinyl estradiol.

6. A method of treating a woman in order to prevent pregnancy or conception without affecting blood pressure, said method comprising the steps of:
a) administering to said woman daily a combination of 2.0 mg of dienogest and 0.030 mg of ethinyl estradiol for at least n×21 days, wherein n=2, 3, 4, 5, 6, 7, 8, 9, 10, 11, 12, 13, 14, 15, 16, and 17; and
b) after the administering of said combination for said at least n×21 days, administering daily to said woman a placebo or no active contraceptive agent for at most 7 days; and then
c) repeating steps a) and b) for a time period during which pregnancy or conception is to be prevented.

7. The method as defined in claim 6, wherein said combination of 2.0 mg of said dienogest and 0.030 mg of said ethinyl estradiol is administered daily to said woman for 84 days and thereafter said placebo or no active contraceptive agent is administered daily to said woman for 7 days during a 91-day cycle.
